(19) United States
(12) Patent Application Publication
(10) Pub. No.: US 2021/0169576 A1
(43) Pub. Date: Jun. 10, 2021

(54) SYSTEM AND METHOD FOR IDENTIFYING COMPARABLE CASES IN PREOPERATIVE SURGICAL PLANNING

(71) Applicant: Ceevra, Inc., San Francisco, CA (US)

(72) Inventors: Russell Kenji Yoshinaka, Lafayette, CA (US); Kenneth Alan Koster, San Francisco, CA (US); Hanna Katherine Winter, San Francisco, CA (US); Ran Berzon, Oakland, CA (US)

(21) Appl. No.: 17/250,572

(22) PCT Filed: Aug. 5, 2019
(86) PCT No.: PCT/US2019/045129

§ 371 Date: Feb. 3, 2021

Related U.S. Application Data
(60) Provisional application No. 62/716,257, filed on Aug. 8, 2018.

(51) Int. Cl. A61B 34/10; A61B 34/00; G16H 10/60; G06N 20/00; A61B 90/00

(52) U.S. Cl. CPC A61B 34/10; A61B 34/25; G16H 10/60; A61B 2090/3762; A61B 90/37; A61B 2034/256; A61B 2090/374; G06N 20/00

(57) ABSTRACT

Systems and methods are provided for identifying comparable cases to assist in preoperative surgical planning Metadata and medical images of a patient are provided for a surgical case, and one or more comparable cases are automatically identified. The metadata can includes patient information and the medical images include any relevant medical image such as a CT scan, MRI image, ultrasound image, and/or three-dimensional reconstructions of any of the foregoing. The one or more comparable cases specify surgical techniques, and analytics or recommendations can be presented from an analysis of the specified surgical techniques based on an analysis of the metadata and the medical image of the surgical case.

### INCORPORATION BY REFERENCE

A PCT Request Form is filed concurrently with this specification as part of the present application. Each application that the present application claims benefit of priority to as identified in the concurrently filed PCT Request Form is hereby incorporated by reference herein in its entirety and for all purposes.

### BACKGROUND

Millions of surgeries are performed every year to treat diverse health conditions including injuries, malignancy, infections, diseases, and many other problems in a patient. These surgeries can carry enormous risk and require a high amount of skill, coordination, knowledge, and resources. Surgeons consider patient-specific anatomical information and medical images from diagnostic procedures (e.g., x-ray, CT, MRI) before performing surgical procedures. Surgeons rely on extensive study, training, and expertise in planning surgical procedures.

Many factors may be considered in preoperative surgical planning, some of which may significantly influence surgical plans. Those factors may render some surgical procedures to be more appropriate than others in different circumstances. In addition, modern advancements in medical technology have opened up more surgical planning options in surgical planning for safer and more effective surgeries. Surgeons must typically balance numerous factors, options, risks, and benefits in preoperative surgical planning with limited access to knowledge regarding prior similar cases and their outcomes.

### SUMMARY

Provided herein is a surgical planning system for identifying surgical cases comparable to a surgery to be performed on a patient. The surgical planning system comprises a database system configurable to receive, from a computing device, (i) patient information about the patient and/or (ii) an image showing a region of the patient where the surgery is to be performed, access a plurality of database records each including a case profile associated with a surgical case, and identify one or more comparable cases, from among the case profiles, where the one or more comparable cases identify one or more surgical techniques and resulting outcomes found in case profiles having similar characteristics with the patient information about the patient and the image showing the region of the patient where the surgery is to be performed.

In some implementations, the database system is further configurable to cause the computing device to present, via a user interface, analytics of the one or more comparable cases based at least in part on the one or more surgical techniques and the resulting outcomes associated with the one or more comparable cases. In some implementations, the database system is further configurable to cause the computing device to present, via a user interface, a recommendation of a surgical technique based at least in part on an analysis of the one or more surgical techniques identified in the one or more comparable cases and the resulting outcomes associated with the one or more comparable cases. In some implementations, the database system is further configurable to receive, from a user input, an indication of a selection of a recommended surgical technique for the surgery to be performed on the patient, and train a machine learning algorithm of the surgical planning system for presenting a recommended surgical technique. In some implementations, the database system is further configurable to receive, from a user input, an indication of a selection of a comparable case to the surgery to be performed on the patient, and train a machine learning algorithm of the surgical planning system for identifying comparable cases. In some implementations, the database system configurable to identify one or more comparable cases is configurable to determine, using a distance function, that the one or more comparable cases have case profiles that are similar to the patient information about the patient and the image showing the region of the patient where the surgery is to be performed. In some implementations, the surgical techniques each include a description of at least a surgery type, surgical procedure, surgical approach, and secondary surgical decisions in performing a surgical operation. In some implementations, the image showing the region where surgery is to be performed includes a medical image of a computed tomography (CT) scan, a magnetic imaging resonance (MRI) scan, an X-ray image, a positron emission tomography (PET) scan, an ultrasound image, and/or a three-dimensional reconstruction of the foregoing. In some implementations, the patient information about the patient includes patient age, gender, weight, height, race, body mass index (BMI), comorbidity status, prior surgical history, or combinations thereof. In some implementations, the patient information further includes tumor size, tumor location, tumor orientation, tumor proximity to organs and/or tissue, tumor growth pattern, or combinations thereof. In some implementations, the case profile includes for each database record postoperative information and patient follow-up information, where the postoperative information and patient follow-up information include: operative time, blood loss, ischemia time, complications, readmissions, length-of-stay in hospital after surgery, positive margins, postoperative erectile function, postoperative continence, renal volume, renal function, or combinations thereof.

Another aspect involves a method for identifying surgical cases comparable to a surgery to be performed on a patient. The method comprises: receiving, from a computing device, (i) patient information about the patient and/or (ii) an image showing a region of the patient where the surgery is to be performed, accessing a plurality of database records each including a case profile associated with a surgical case, and identifying one or more comparable cases, from among the case profiles, where the one or more comparable cases identify one or more surgical techniques and resulting outcomes found in case profiles having similar characteristics with the patient information about the patient and the image showing the region of the patient where the surgery is to be performed.

In some implementations, the method further comprises causing the computing device to present, via a user interface, analytics of the one or more comparable cases based at least in part on the one or more surgical techniques and the resulting outcomes associated with the one or more comparable cases. In some implementations, the method further comprises causing the computing device to present, via a user interface, a recommendation of a surgical technique based at least in part on an analysis of the one or more surgical techniques identified in the one or more comparable cases and the resulting outcomes associated with the one or more comparable cases. In some implementations, the method further comprises receiving, from a user input, an indication of a selection of a comparable case or a recommended surgical technique to the surgery to be performed on the patient, and training a machine learning algorithm of the surgical planning system. In some implementations, identifying the one or more comparable cases includes determining, using a distance function, that the one or more comparable cases have case profiles that are similar to the patient information about the patient and the image showing the region of the patient where the surgery is to be performed. In some implementations, the surgical techniques each include a description of at least a surgery type, surgical procedure, surgical approach, and secondary surgical decisions in performing a surgical operation. In some implementations, the image showing the region where surgery is to be performed includes a medical image of a computed tomography (CT) scan, a magnetic imaging resonance (MRI) scan, an X-ray image, a positron emission tomography (PET) scan, an ultrasound image, and/or a three-dimensional reconstruction of the foregoing. In some implementations, the patient information about the patient includes patient age, gender, weight, height, race, body mass index, comorbidity status, prior surgical history, tumor size, tumor location, tumor orientation, tumor proximity to organs and/or tissue, tumor growth pattern, or combinations thereof. In some implementations, the case profile includes for each database record includes postoperative information and patient follow-up information, where the postoperative information and patient follow-up information includes: operative time, blood loss, ischemia time, complications, readmissions, length-of-stay in hospital after surgery, positive margins, postoperative erectile function, postoperative continence, renal volume, renal function, or combinations thereof.

### BRIEF DESCRIPTION OF THE DRAWINGS

FIG. 1 shows a schematic diagram of an example environment including a user device in communication with a database system of a surgical planning system according to some implementations.

FIG. 2A shows a schematic diagram of an example database record of a case profile according to some implementations.

FIG. 2B shows an image of an example three-dimensional reconstruction of a patient's kidney and adjacent blood vessels according to some implementations.

FIG. 3 shows a flow diagram of an example method for identifying surgical cases comparable to a surgery to be performed on a patient according to some implementations.

FIG. 4 shows an image of an example user interface displaying patient information for a surgical case according to some implementations.

FIG. 5 shows an image of an example user interface displaying criteria in a search query for comparable cases according to some implementations.

FIG. 6 shows an image of an example user interface displaying analytics for surgical techniques associated with comparable cases according to some implementations.

FIG. 7 shows an image of an example user interface displaying scores for surgical techniques associated with comparable cases according to some other implementations.

### DETAILED DESCRIPTION

### I. Introduction

Various embodiments of this disclosure pertain to systems and methods that identify comparable cases to assist in planning a new surgery, present analytics on comparable cases, present recommendations of a surgical plan, and/or score a proposed surgical plan. A surgical planning system may use one or both of metadata and medical images to automatically identify comparable cases to the surgery case being planned. In certain embodiments, a surgeon or other individual or entity (e.g., team, hospital, corporation, etc.) responsible for planning a surgery selects or enters parameters that describe the surgery case being planned, e.g., patient age, gender, size of tumor, surgery type. The individual or entity may enter or identify one or more images for the surgery case, or such images may be automatically identified by a computing device. Images to be identified or entered may include any relevant medical image, including a CT scan, MRI image, PET image, x-ray image, ultrasound image, and a three-dimensional reconstruction of any of the foregoing. With the input parameters and image(s), a surgical planning system identifies one or more comparable cases each including a surgical technique and/or approach, and optionally generates and presents analytics associated with the comparable cases.

### II. Terminology

As used herein, the term “surgery” refers to a procedure for treating injuries, disorders, and other medical conditions by incision and associated manipulation, particularly with surgical instruments. The terms “operation,” “surgical operation,” and “surgery” are used throughout this disclosure. Unless otherwise clear from context, the terms are used interchangeably.

As used herein, the term “surgical case” refers to a patient-specific medical condition that has been or will be treated by surgery. A surgical case is described by various types of metadata, sometimes referred to as patient information. A surgical case may be stored as a database record or other accessible record of the case.

As used herein, the term “metadata” is data represented in a textual and/or numerical format that characterizes an item such as a surgical case. In various embodiments, metadata contains patient information relevant to preoperative surgical planning. Various types of data and/or metadata may include basic information, preoperative parameters, surgical technique, postoperative information, and patient follow-up information, which are described in more detail below.

As used herein, a “case profile” refers to a summary of a surgical case that includes some or all of the metadata identified above, and optionally additional information such as a medical image, the associated radiology report, a video of the operation and/or a three-dimensional reconstruction of the medical image. In some cases, case profiles are stored as data records and used in searching for comparable cases and/or returned as full or partial results for each comparable case identified in a search.

As used herein, a “comparable case” refers to a surgical case performed in the past that shares some degree of similar characteristics to a surgical case for which an operation is being planned. Similar characteristics may include various types of overlapping information or factors such as (a) patient demographics (e.g., age, gender, race, body mass index (“BMI”)), (b) diagnosis (e.g., renal cell carcinoma), and (c) characteristics of the patient's anatomy (e.g., size of tumor, location of the tumor, number of kidneys, number of arteries and blood vessels, whether the tumor has invaded a blood vessel). Similar characteristics may also include similarity of images. For example, a comparable case may have images (e.g., CT scan or three-dimensional model) in which the tumor has very similar size and location, and/or proximity to another structure of interest such as a blood vessel or another organ.

A determination of what makes a prior surgical case sufficiently similar to a future surgical case to qualify as a comparable case may be made using human-calibrated algorithms and/or machine learning. Various search algorithms may be employed to identify comparable cases from among records of many other cases stored in a database system or other data repository. Examples include distance functions of various types, filters that exclude surgical cases outside of some threshold for a particular type of information, and/or models that select surgical cases using conditional logic, computational networks, regression analysis, etc.

As used herein, a “surgical planning system” refers to software or other computational logic for performing one or more functions associated with surgical planning. Such functions may include identifying comparable cases, performing analytics on comparable cases, recommending a surgical technique or one or more aspects thereof, scoring a proposed surgical technique or one or more aspects thereof, identifying factors that have a strong bearing on the outcome of a particular surgical technique or one or more aspects thereof, and the like. As used herein, a “surgical technique” may include: a surgery type, a surgical procedure, a surgical approach, and/or secondary surgical decision(s) as described in more detail in paragraph [0039] below. A surgical planning system may contain or be configured to interact with a database or databases containing surgical case information or case profiles. In some implementations, the logic for identifying comparable cases and/or performing other functions may reside and/or execute on one or more remote servers, on a user device, or on computing device that may serve as an intermediary between a remote server and the user device.

As used herein, a “search” refers to a computational process to locate a data record or other information meeting defined criteria, which criteria are sometimes referred to as a search query. In certain embodiments, a search is used to locate comparable cases for patient information and/or images of tissue, bone, or organ on which surgery will be performed. A search may be limited by certain filters. For example, a search criterion may require locating only comparable cases for individuals of the same gender as the patient for the surgical case being searched. In another example, a search criterion may require locating only comparable cases for individuals having ages within two years of the age of the patient.

As used herein, a “distance function” refers to a logical or mathematical representation of distance between surgical cases, typically between two surgical cases such as between a pre-existing surgical case (a potential comparable case) and a current surgical case. The distance may be provided in a multidimensional space in which dimensions represent patient information such as BMI, surgery type, gender, age, and information about images of the patient's tissue, bone, or organ on which surgery will be performed. In other words, each surgical case is presented as a point in the multidimensional space. Any two such surgical cases can be characterized by a degree of similarity based on their separation in the multidimensional space. In a simple case, the separation distance (or degree of similarity) is determined based on a difference in the position of the points, e.g., a Euclidean distance. It will be understood that many other distance metrics may be employed such as those that weigh some dimensions more than others (e.g., a distance function may weigh tumor size more heavily than patient gender). Distance functions may have non-geometric representations such as conditional logic or classification trees.

As used herein, “preoperative surgical planning” refers to a process of determining a surgical technique that is intended to be employed during a surgery. Preoperative surgical planning primarily involves an evaluation of the patient's condition, medical record, and imaging, but may also involve analyzing comparable cases and evaluating analytics and/or recommendations based on that analysis.

As used herein, an “image” refers to a visible depiction or other representation of an item such as the abdominal area of a patient. In various embodiments presented herein, images provide representations of morphologies and/or compositions of tissue, blood vessels, bone, or organs in a subject. Such images are sometimes referred to herein as medical images. Medical images of varying modality include without limitation representations of tissue, blood vessels, bone, and/or organs derived from computed tomography (CT), magnetic resonance imaging (MRI), x-ray imaging, positron emission tomography (PET), ultrasound imaging, and two-dimensional and/or three-dimensional reconstructions of any of the foregoing.

As used herein, “analytics” refer to analyses and associated presentation of information about surgical cases. Often, analytics present information that facilitates decisions about a surgical plan. In some cases, analytics present information regarding multiple comparable cases or cases meeting some other criteria associated with the surgery under consideration. In certain embodiments, the analytics are presented on an aggregated basis. For example, analytics may compare multiple surgical approaches based on the characteristics of the case being planned. In another embodiment, analytics may compare multiple surgical approaches based on a set of desired outcomes. In another embodiment, average metrics (e.g., operative time, blood loss) for a chosen surgical approach are presented for different surgical approaches.

### III. Case Information Input to Surgical Planning System

Doctors, nurses, caretakers, hospitals, clinics, or other healthcare professionals may create and maintain detailed records of patients that have been or will be treated surgically. Data regarding the patient and the patient's condition may be recorded and stored electronically. Data regarding the patient and the patient's condition may come from, for example, electronic medical records (EMRs), medical evaluations, medical examination histories, lab test reports, pathology reports, biopsy reports, radiology reports, computed tomography (CT) scans, magnetic resonance imaging (MRI) images, x-ray images, and/or other sources. The data may be organized in various fields or categories, and may exist in one or more database records in one or more database systems.

When a surgery is being planned, patient information or case information may be provided. The patient information may be entered into the surgical planning system or retrieved from a database or databases. The patient information may be provided for any of various purposes, such as allowing the surgical planning system to identify comparable cases, make recommendations for surgical plans, provide training to refine algorithms used to identify comparable cases and/or make recommendations, and the like. In some implementations, the patient information is entered or selected in textual and/or numerical format, where the patient information describes the surgical case being planned, e.g., patient age, gender, comorbidity status, BMI, tumor size, surgery type, etc.

In some implementations, medical images may be provided in addition to patient information and entered into the surgical planning system or retrieved from a database or databases. In certain embodiments, some medical images may be stored and archived in a database or databases configured for storage and maintenance of medical images and medical imaging data. For example, such a database may be referred to as a picture archiving and communication system (PACS), where PACS may have CT images, MRI images, or other source images. In some cases, the medical images are stored on physical media such as CD/DVD/external hard drives, and sometimes they are stored on hospital devices such as a computer.

In some implementations, medical images may be processed prior to use in a search or search query. Medical images may be processed in one or more ways to enable comparison between images from different cases or to facilitate the extraction of information used to compare cases. Example processing mechanisms may include image segmentation (using manual, automatic, or a combination of manual and automatic means), image registration (the alignment of multiple images into a single integrated image, often by matching up visual features present in individual images), analysis of image annotations (such as bounding boxes surrounding lesions, arrows pointing at image areas of interest, etc.), image processing (Gaussian blur, image sharpening, etc.), gradient detection, measurements of volumes, and measurements of distances and/or angles between features in images.

Patient information and/or images may be provided in one or more database records to create a case profile. The patient information and/or images may be provided from a user device or retrieved from a database or databases. The surgical planning system of the present disclosure leverages the patient information and/or images of the surgical case to search for and identify comparable cases, make recommendations for surgical plans, provide training to refine algorithms used to identify comparable cases and/or make recommendations, and the like.

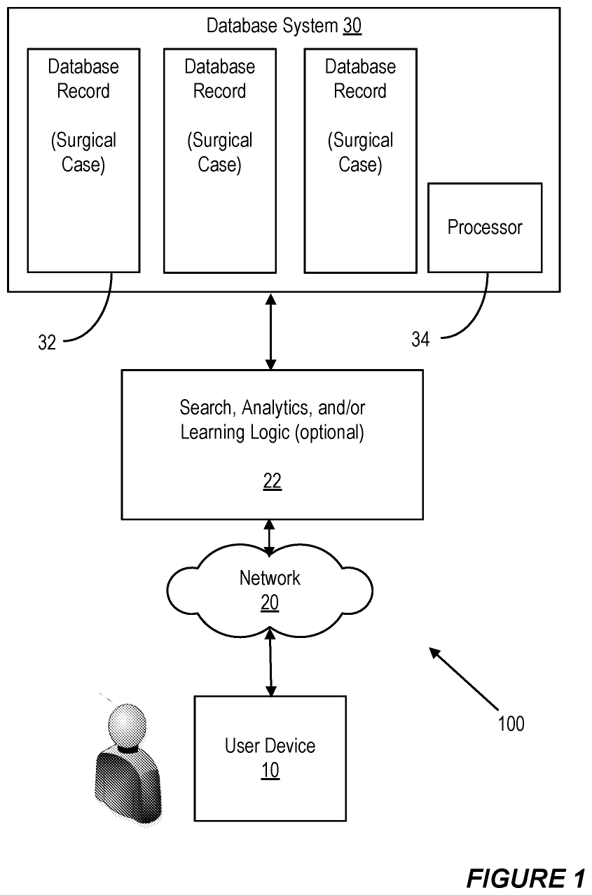

FIG. 1 shows a schematic diagram of an example environment including a user device in communication with a database system of a surgical planning system according to some implementations. An environment 100 includes a database system 30 and a user device 10 that communicates with the database system 30 over a network 20. The user device 10 can be any computing device that can access the database system 30. For example, the user device 10 can be a mobile phone (e.g., smartphone), tablet, laptop computer, desktop computer, work station, or other computing device capable of interfacing directly or indirectly with the database system 30. Each user device 10 typically includes a display such as a monitor screen, liquid crystal display (LCD), and light-emitting diode (LED) display, among other possibilities. Data, pages, forms, applications, media content, or other information provided by the database system 30 may be presented in a user interface through the display.

The database system 30 includes various hardware and software elements of the surgical planning system. The database system 30 may include one or more servers, which may be dedicated or leased such as via cloud computing resources. Data is stored in the database system 30 and may be accessible by one or more users of the user device 10. Data may be stored in different objects such as database records 32. The database system 30 may handle creation, storage, organization, and/or access to the database records 32. Case profiles of various surgical cases may be stored as database records 32 or other accessible record. The database system 30 includes a processor 34 for implementing various processes and functions of the database system 30. In some embodiments, the processor 34 includes program code for executing logic for searching, identifying, analytics, and/or learning operations of the surgical planning system. It will be understood that the processor 34 includes program code for executing logic for some of the aforementioned operations and not others. By way of one example, the program code may execute logic for identifying comparable cases but not for performing analytics and learning. By way of another example, the program code may execute logic for identifying comparable cases and learning but not for performing analytics. By way of another example, the program code may execute logic for identifying comparable cases and for performing analytics but not for learning. Logic for other operations not executed by program code in the processor 34 may be implemented in the user device 10 and/or intermediaries between the database system 30 and the user device 10.

The user device 10 may communicate with the database system 30 using the network 20, where the network 20 can include one or any combination of a LAN (local area network), WAN (wide area network), telephone network, wireless network, cellular network, point-to-point network, star network, token ring network, hub network, or other appropriate configuration. For example, the network 20 can include a TCP/IP network through which data may be communicated between the database system 30 and the user device 10. In some embodiments, requests such as search queries may be inputted from the user device 10 and received by the database system 30, and relevant surgical cases in one or more database records 32 may be identified and presented from the database system 30 to the user device 10.

In some embodiments, the environment 100 may further include a search, analytics, and/or learning logic 22 that may execute operations of the surgical planning system independent of the database system 30. The database system 30 may serve as a repository for surgical cases and the search, analytics, and/or learning logic 22 may interact with the database system 30 to access and process appropriate data from the database system 30. The search, analytics, and/or learning logic 22 describes computational and algorithmic operations in software that may be installed and running on one or more servers of the database system 30, installed and running on one or more intermediaries between the user device 10 and the database system 30, and/or installed and running on the user device 10.

In some embodiments, doctors, nurses, caretakers, hospitals, clinics, or other healthcare professionals provide patient information and medical images by manually entering or uploading such information and medical images into the database system 30 of the surgical planning system. In some embodiments, there is an integration between the database system 30 of the surgical planning system and other database systems (not shown). Other such database systems may include a hospital image storage system, which is sometimes referred to as a picture archiving and communications system (PACS). This allows the database system 30 of the surgical planning system to automatically pull CT scans, MRI images, or other image from the hospital image storage system once patient information about the patient is provided to the database system 30 of the surgical planning system. For example, once a patient's medical record number (MRN) is provided, the patient's CT/MRI images may be retrieved from the hospital image storage system and provided to the database system 30 of the surgical planning system. In some implementations, once the patient's CT/MRI images are retrieved, the database system 30 of the surgical planning system may be utilized to create a three-dimensional reconstruction of the CT/MRI image. In some implementations, the surgical planning system may create the three-dimensional reconstruction either automatically or with some degree of human intervention.

In some embodiments, other such database systems (not shown) integrated with the database system 30 of the surgical planning system may include an electronic medical record (EMR) system and/or PACS. This allows the database system 30 of the surgical planning system to retrieve relevant metadata about the patient, and/or medical imaging for the patient, when certain patient information is provided. Doctors, nurses, caretakers, hospitals, clinics, or other healthcare professionals may provide certain patient information, e.g., a patient's MRN, to the database system 30 of the surgical planning system, and the patient's medical history and prior medical examinations may be retrieved from the EMR system, and the patient's prior medical imaging my be retrieved from the PACS, and provided to the database system 30 of the surgical planning system.

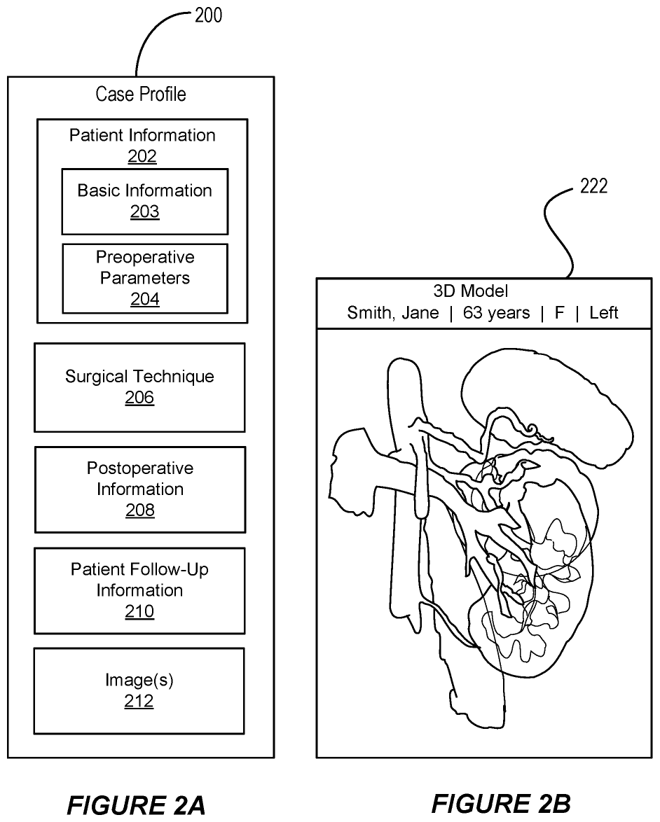

FIG. 2A shows a schematic diagram of an example database record of a case profile according to some implementations. A case profile may be created when a patient with a medical condition has been treated by surgery or will be treated by surgery. A case profile 200 of a surgical case is described by various types of metadata and/or media content. In some embodiments, the media content of the case profile 200 may include images and/or videos. In some embodiments, the various types of metadata and/or media content may be accessible in a single database, which may access data stored on one or more physical mediums. In some embodiments, the various types of metadata and/or media content may be accessible across multiple databases. For example, some basic information, diagnostic images, and/or video recordings of a surgery may be sourced from across multiple databases or spread across multiple databases. Nonetheless, the multiple databases may be shared in an integrated database system so that the various types of metadata and/or media content are accessible by the user.

The various types of metadata and/or media content specific to a patient that has been or will be treated by surgery may be stored and associated with a case profile 200. Types of data and metadata associated with the case profile 200 can include but are not limited to patient information 202 (including basic information 203 and preoperative parameters 204), surgical technique 206, postoperative information 208, patient follow-up information 210, and image(s) 212.

The basic information 203 pertains to general information about the patient that has been or will be treated by a surgery. Example parameters of the basic information 203 can include patient identification information such as patient name and medical record number (“MRN”). Additional or alternative parameters of the basic information 203 can include patient demographics information such as gender, race, age, weight, height, and BMI. Other example parameters of the basic information 203 can include patient medical information such as comorbidity status, prior operations or treatments, and medical history. Other example parameters of the basic information 203 can include surgical information such as surgery type (e.g., partial nephrectomy), surgery date, and surgeon name.

The preoperative parameters 204 relate to information about the patient's medical condition that was treated or will be treated by surgery. An example parameter of the preoperative parameters 204 includes patient diagnosis (e.g., renal cell carcinoma). Additional or alternative parameters of the preoperative parameters 204 can include characteristics of the patient anatomy and/or tumor, which can include type of organs affected, number of organs affected, tumor size, tumor location, tumor orientation, tumor proximity to organs and/or tissue, tumor growth pattern, and number of blood vessels potentially affected by the surgery. Other example parameters of the preoperative parameters 204 can include expected complexity of the surgery and results of lab tests. Sources of the preoperative parameters 204 can be taken from, for example, radiology reports, pathology reports, CT scans, lab test reports, MRI images, and/or other image information.

The surgical technique 206 includes information about a contemplated or actual surgery performed on a patient. In certain embodiments, the surgical technique 206 contains information that describes the type of surgery and the manner in which it was performed. Such information may include a surgery type, a surgical procedure, a surgical approach, other surgery details, or combinations thereof. Surgery type refers to the general type of surgery performed. Examples include but are not limited to radical nephrectomy (removing an entire kidney that contains a tumor), partial nephrectomy (removing a tumor from a kidney and some of the surrounding renal tissue but preserving the kidney itself), donor nephrectomy (transplanting a kidney from a donor to a recipient), radical prostatectomy (removing an entire prostate that contains a tumor), appendectomy (removing the appendix), tonsillectomy (removing the tonsils), knee/hip replacement, pulmonary lobectomy (removing a lobe of a lung), pulmonary wedge resection (removing a portion of a lung, not amounting to an entire lobe), and other types of surgeries. Surgical procedure describes the manner in which the surgery is performed, which may be described in terms of invasiveness and equipment used. Examples include laparoscopic, open, robotic-assisted laparoscopic, video-assisted thoracoscopic (VATS), robotic-assisted thoracoscopic, and keyhole (neurosurgery). Surgical approach describes the route taken by the surgeon to access the operative area. For example, one of the surgical approaches involve whether to approach the affected region (e.g., kidney) from the front (“transperitoneal”) or from the rear (“retroperitoneal”), and other surgical approaches can involve endonasal endoscopic, mini-pterional, retromastoid, and supra-orbital eyebrow (brain surgery). Other specific decisions in describing surgical technique, which may be referred to as secondary surgical decisions, can include but are not limited to which blood vessels to clamp, incision depth, whether a robotic arm is used during the surgery, and what types of imaging modalities are used during the surgery.

The postoperative information 208 includes information about the direct results of the surgery. Such information regarding the outcome of the surgery can be recorded as soon as the surgery is completed. Examples of postoperative information 208 include operative time, blood loss, ischemia time, intraoperative complications, mortality, intraoperative conversion from one surgery type to another (e.g., convert from partial nephrectomy to radical nephrectomy), and intraoperative conversion from one surgical procedure to another (e.g., convert from laparoscopic to open).

The patient follow-up information 210 includes information about the indirect results or later results of the surgery. Such information regarding the outcome of the surgery can be recorded after a time period (e.g., 2 months) from which the surgery is completed. It will be understood that the time period can vary depending on the patient's circumstances. Examples of patient follow-up information 210 include postoperative length of hospital stay, margin status, postoperative complications, postoperative readmissions, postoperative erectile function, postoperative continence, renal volume, renal function, and the like.

The images 212 provide representations of morphologies and/or compositions of tissue, blood vessels, bone, or organs in a subject. The images 212 may provide a visual depiction of an affected area (e.g., abdominal area) in which surgery is to be performed. The images 212 are also referred to as medical images. Medical images of varying modality include without limitation representations of tissue, bone, and/or organs derived from CT, MRI, x-ray imaging, PET, ultrasound imaging, and two-dimensional and/or three-dimensional reconstructions of any of the foregoing. In some implementations, the images 212 may be provided preoperatively such as for diagnostic purposes. In some implementations, various images may be accessed from a database of medical images (e.g., PACS), and the images 212 related to the patient may be associated with the database record of the case profile 200. The images 212 may be processed in one or more ways to facilitate extraction of information used to compare cases. The images 212 may be processed in one or more ways to create two-dimensional or three-dimensional reconstructions of an affected area. In some implementations, the images 212 may be used during a surgical operation (i.e., intraoperatively).

FIG. 2B shows an image of an example three-dimensional reconstruction of a patient's kidney and adjacent blood vessels according to some implementations. The image 222 is a three-dimensional reconstruction of an affected area of a patient that may be created from cross-sectional images such as CT scans and MRI images. Three-dimensional models of tissue, blood vessels, bone, and/or organs may be created by converting two-dimensional images from conventional medical imaging sources. The image 222 may be associated with a particular patient for a specific surgical case. The image 222 may be displayed with metadata of the specific surgical case, such as the patient's name, age, gender, and kidney side. The image 222 may be processed and to extract information to be used in comparing cases.

### IV. Searching for Comparable Cases

A surgical planning system may employ metadata and/or medical images to identify comparable cases to the surgical case being planned. In various embodiments, the surgical planning system employs both metadata and medical images as inputs to identify comparable cases. In some embodiments, a user inputs metadata and/or medical images into a user interface of a computing device (such as user device 10 in FIG. 1) as search parameters. In some embodiments, a user inputs some metadata into a user interface of a computing device (such as user device 10 in FIG. 1) as search parameters, and additional metadata and medical images are retrieved from one or more databases and provided to the surgical planning system.

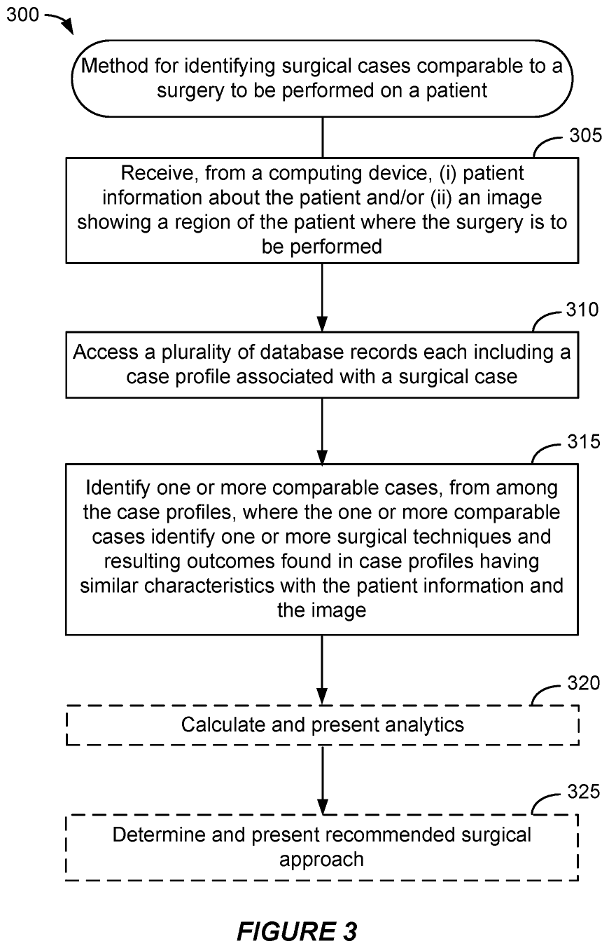

FIG. 3 shows a flow diagram of an example method for identifying surgical cases comparable to a surgery to be performed on a patient according to some implementations. The process 300 may be performed in a different order or with different, fewer, or additional operations.

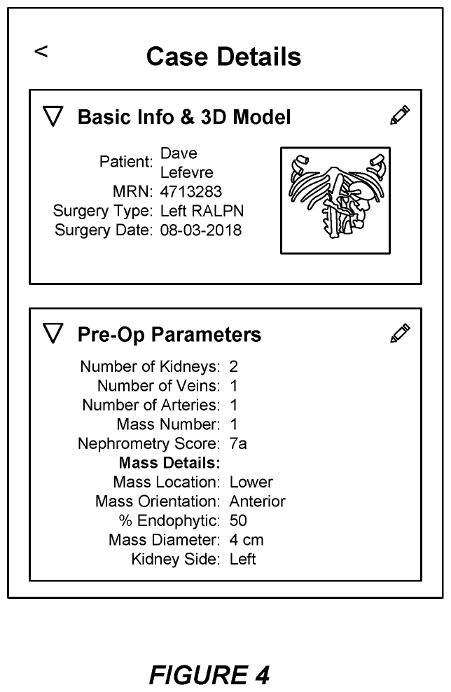

At block 305 of the process 300, (i) patient information about the patient and/or (ii) an image showing a region of the patient where the surgery is to be performed are received from a computing device. In some implementations, both (i) patient information about the patient and (ii) an image showing a region of the patient where surgery is to be performed are received from a computing device. Patient information includes basic information and preoperative parameters as described above in FIG. 2A and as shown in FIG. 4. In some implementations, patient information can include information about the image, which can be obtained from an analysis of the image. For example, information about the image can include but is not limited to tumor size, tumor location, tumor orientation, tumor proximity to organs and/or tissue, tumor growth pattern, and number of blood vessels potentially affected by the surgery.

FIG. 4 shows an image of an example user interface displaying patient information for a surgical case according to some implementations. In FIG. 4, the user interface displays basic information including the patient's name, patient's MRN, patient's surgery type, and surgery date. The user interface also displays preoperative parameters including the number of kidneys, number of veins, number of arteries, mass number, and nephrectomy score (which indicates the complexity of the surgery). Furthermore, the preoperative parameters include the mass characteristics such as the mass location, mass orientation, percentage of endophyticity, mass diameter, and kidney side.

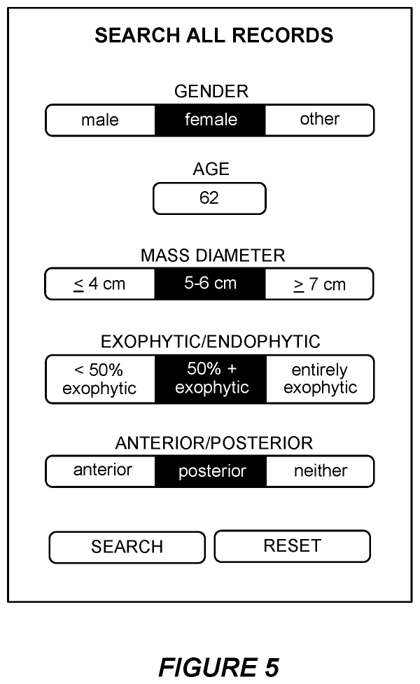

As described above, some of the patient information may be entered or selected as user inputs into a user interface. FIG. 5 shows an image of an example user interface displaying metadata inputs in a search query for comparable cases according to some implementations. FIG. 5 shows metadata inputs for a comparable case search query. The metadata inputs in FIG. 5 include gender, age, mass diameter, percentage of endophyticity/exophyticity, and tumor location. In some implementations, the metadata inputs are used in a search query to identify surgical cases having case profiles that meet the parameters of the search query or within a threshold for certain types of information. In some implementations, some or all of the information from the metadata inputs are used by the surgical planning system in a search for comparable cases.

In some embodiments, images are not provided with the patient information to locate one or more comparable cases. In other words, only the patient information (in the form of metadata) is leveraged as part of the search for one or more comparable cases. However, in some embodiments, images are provided with the patient information to locate one or more comparable cases. For example, the images may be manually entered or uploaded with the patient information to perform the search query. Doctors, nurses, caretakers, other healthcare professionals, hospitals, clinics, or other entities may create a partial or complete patient information record by entering (manually and/or automatically) basic information for the case and source data such as CT or other images, radiology reports, etc. In some embodiments, the source data such as CT or other images, radiology reports, etc., may be automatically retrieved upon entry of the basic information. The doctor, nurse, caretaker, other healthcare professional, hospital, clinic, or other entity may provide a three-dimensional model of the image(s), which is then associated with the surgical case or case profile. The surgical planning system uses this patient information and/or images to search for comparable cases. In some embodiments, searching for comparable cases and/or providing a recommended surgical plan is performed using a model such as a trained neural network, a classification tree, a random forest model, etc. that has been trained using experience from surgical cases provided to a training algorithm.

In some implementations, the image(s) may be processed prior to search to extract image information to enable comparison between the image(s) and one or more images from other cases. The image(s) may be processed by image segmentation, image registration, analysis of image annotations, image processing, gradient detection, measurements of volumes, and measurements of distances and/or angles between features in images. In some embodiments, the surgical planning system may process the image(s) automatically or with some degree of human intervention.

At block 310 of the process 300, a plurality of database records, each including a case profile associated with a surgical case, are accessed. Each of the database records may relate to prior surgical cases of various patients, where the database records may be stored and maintained across one or more databases. The case profiles include patient information, surgical technique, postoperative information, patient follow-up information, and image(s) as shown in FIG. 2A. One or more comparable cases identified at block 315 are drawn from the plurality of database records at block 310.

At block 315 of the process 300, one or more comparable cases are identified from among the case profiles. The one or more comparable cases identify one or more surgical techniques and resulting outcomes found in case profiles having similar characteristics to the patient information about the patient and the image showing the region of the patient where surgery is to be performed. In some implementations, identification of one or more comparable cases can be determined using a distance function. In certain embodiments, each element of metadata and each feature extracted from image processing constitutes a single dimension in a multidimensional space of surgical cases. For example, for partial nephrectomy case types, the tumor size can be one dimension, the kidney side can be a second dimension, the number of renal arteries can be a third dimension, the patient age can be a fourth dimension, and so on. The distance function calculates a distance component for each relevant dimension of two surgical cases being compared, and then combines those components to determine a total distance. An example calculation of component distances for individual dimensions is subtraction, while example combinations of component distances can utilize addition, weighted multiplication (where more “important” components are assigned larger weights), Euclidean distances, or non-linear functions employing conditional logic.

In certain embodiments, searching for comparable cases does not rely entirely on using a distance function, but instead includes using models that identify comparable cases based on input patient information, such as the patient metadata and images described above. Such models may be neural networks, etc. that have been trained to identify comparable cases and/or, in some implementations, recommend surgical plans based on input patient information.

Metadata and images for comparable cases may be stored within databases, data files on local persistent storage devices, distributed shared memory, network-attached file storage, network accessible file storage, or storage managed by distributed storage/computer clusters (e.g., Hadoop). In some implementations, the metadata and/or images for the comparable cases were input at appropriate times. Hospital records are one source of the metadata and/or images. In some implementations, the metadata and/or images were collected by and/or retrieved from existing records by entities associated with providing or maintaining the database system of the surgical planning system. In some embodiments, the metadata may be collected into and retrievable from an EMR system. In some embodiments, the images may be collected into and retrievable from a hospital imaging storage system (e.g., PACS). Surgical case information for the comparable cases may be constructed from the various data sources of the metadata and/or images of prior surgeries.

### V. Comparable Case Results from the Surgical Planning System

The one or more identified comparable cases at block 315 may be provided as data to the user in one of several ways. The data may be presented to the user in the form of an unaggregated list of comparable cases and associated case information (e.g., patient information, preoperative parameters and surgical technique), analytics regarding the comparable cases which are presented on an aggregated basis, recommendation of a proposed surgical technique, and/or scoring for proposed surgical techniques.

At block 320 of the process 300, analytics are optionally calculated and presented. The surgical planning system may be designed or configured to provide analytics regarding the one or more comparable cases, or at least cases meeting some other criteria associated with the surgery under consideration. In some embodiments, the analytics are presented on an aggregated basis. In one example, analytics may compare common surgical approaches based on some postoperative information. In another example, the analytics can be shown on a scatter plot with a point per comparable case, where, for example, one axis of the scatter plot is surgical approach, and the other is operative time. In another embodiment, surgical approaches with the “best” outcomes based on the characteristics of the case being planned are compared. In another embodiment, average metrics (e.g., operative time, blood loss) for a chosen surgical technique are presented.

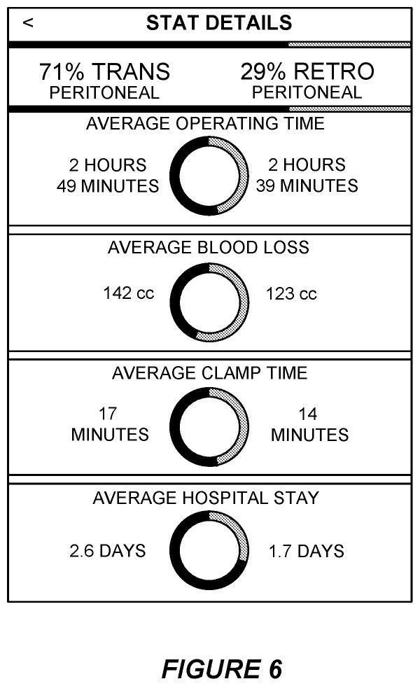

FIG. 6 shows an image of an example user interface displaying analytics for surgical techniques associated with comparable cases according to some implementations. FIG. 6 shows an example of analytics being presented on an aggregated basis. After the surgical planning system identifies comparable cases, the surgical planning system calculates analytics for surgical techniques from the comparable cases. In FIG. 6, analytics are presented for surgical approaches in a partial nephrectomy between transperitoneal and retroperitoneal surgical approaches. The analytics shows that 71% of the comparable cases utilized a transperitoneal surgical approach and that 29% of the comparable cases utilized a retroperitoneal surgical approach. Average operating time, average blood loss, average clamp time, and average hospital stay are calculated for the transperitoneal approaches and for the retroperitoneal approaches.

At block 325 of the process 300, a recommended surgical approach is determined and recommended. The surgical planning system may determine a recommended surgical technique based on its analysis of the metadata and/or medical image for the surgical case being planned. The analysis of the metadata and/or medical image can involve optimizing surgical outcomes for the surgical case being planned. The recommendation is based on an analysis of (a) the various surgical techniques employed in the one or more identified comparable cases and/or (b) the results of such cases, as reflected in the postoperative information and patient follow-up information. The surgical planning system will then determine the surgical technique associated with the “best” surgical outcomes, and present such technique as the recommendation. It will be understood that the “best” outcomes may be dependent on the preferences, desires, or expectations of the user. For example, some users may be willing to have a higher risk of a positive margin after a prostatectomy but be less willing to forfeit sexual function, and some users may be more willing to risk a higher rate of complications in return for a faster recovery period. In certain embodiments, a recommended surgical technique is determined by a model or other algorithm associated with the surgical planning system. As an example, the algorithm may iterate over the domains of each surgical technique dimension for the given surgery type, evaluating the expected outcome of the use of that surgical technique based on, for example, a trained neural net model, a Bayes net model, etc., and selecting the combination or combinations of values across those dimensions that optimize the expected outcomes of the case. While not required in the present disclosure, the surgical planning system can be capable of recommending a preoperative surgical plan by deciding between surgery and another form of treatment such as radiation.

In addition or in the alternative, the surgical planning system may present a score for a proposed surgical plan or scores for multiple surgical plans. The score may be based on an analysis of the metadata and the medical image of the surgical case being planned, and based on an analysis of (a) the surgical techniques performed in each of the one or more identified comparable cases and/or (b) the results of such cases, as reflected in the postoperative information and patient follow-up information. The surgical planning system assigns scores to the surgical techniques that can reflect a “confidence” rating for each of the surgical techniques being proposed. A confidence score is a measure of the statistical likelihood that a particular surgical technique (or one or more aspects of that technique such as a surgical approach or a surgical method used in the technique) will maximize a specified outcome, which may be defined in many ways. Examples of a specified outcome include one or more postoperative criteria such as no recurrence of the condition necessitating the surgery, less than a defined blood loss during the operation, less than a defined length of hospital stay, etc.

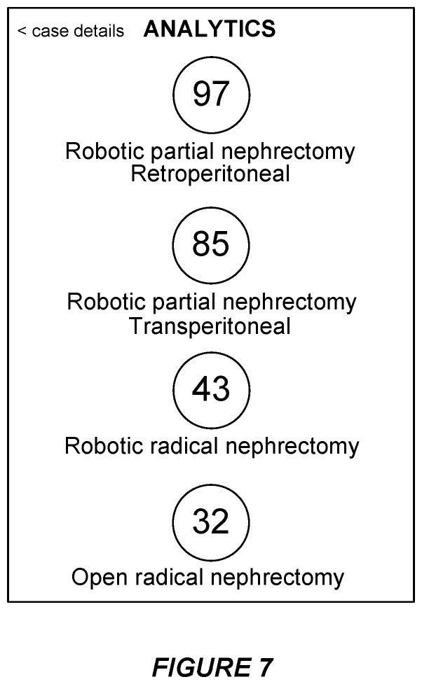

FIG. 7 shows an image of an example user interface displaying scores for surgical techniques associated with comparable cases according to some other implementations. Patient information for John Doe is received by the surgical planning system, and comparable cases are identified by the surgical planning system. The surgical planning system identifies surgical techniques from the comparable cases. In FIG. 7, the comparable cases identified at least four different surgical techniques, including robotic partial nephrectomy retroperitoneal, robotic partial nephrectomy transperitoneal, robotic radical nephrectomy, and open radical nephrectomy. The robotic partial nephrectomy retroperitoneal had a score of 97, the robotic partial nephrectomy transperitoneal had a score of 85, the robotic radical nephrectomy had a score of 43, and the open radical nephrectomy had a score of 32, where each of the scores reflect a confidence rating for the proposed surgical technique based on an analysis of patient information, postoperative parameters and patient follow-up information. From a comparison of the characteristics of John Doe with the comparable cases and assessing the positive outcomes of the surgical techniques identified by the comparable cases, the surgical planning system can provide a confidence rating.

As illustrated in FIG. 7, scoring alternative surgical techniques may give a surgeon confidence that one technique is superior to the others and will have a high likelihood of maximizing a positive outcome. The surgeon may then select that technique for her surgical plan. However, there may be surgical cases in which no surgical technique has a high confidence score. For example, all techniques may have a score of 50 (out of 100) or lower. In such cases, a surgeon may conclude that surgery is not a good option, or that another surgeon should conduct the operation, or that a factor other than confidence score should be used to determine which surgical technique to employ.

### VI. Learning

In some implementations of the process 300, the surgical planning system may learn based on input received from the surgeon, doctor, nurse, hospital, clinic, or other individual or entity. For example, in some implementations of the process 300, comparable cases may be determined via a distance function that applies a weighted average to the component per-dimension distances. Some implementations may record input from an individual or entity, such as a click or a tap on a case, as a positive signal for similarity according to the judgment of the individual or entity. If that positive signal is associated with the case that the system has determined is the “most” comparable, then it is a positive reinforcement for the weights used in the calculation of the distance function; if it is associated with a case which is not the “most” comparable, then it becomes a signal that indicates that weights should be adjusted to favor the case being selected as being determined to be the most comparable. This learning refines search of comparable cases and recommendations.

In certain embodiments, the surgical planning system is trained based on the outcomes of various surgical plans chosen for various surgical cases (or case profiles). For example, a group of surgical cases sharing certain characteristics (e.g., renal cell carcinoma) may together provide a training set for training a model that recommends surgical techniques when presented with new surgical cases falling within the group. The data for the training set may come from, without limitation, historical surgical case data, data accrued through ongoing use of surgical planning system, or synthetic data derived from historical data or data accrued through ongoing use. Within the training set, different surgical techniques provide various outcomes, some of which may be successful, some unsuccessful, and some of mixed success. Of course, success must be defined appropriately for the context. For example, success may be defined based on postoperative recovery time, recurrence rate, ischemia duration, etc. The training set may also include patient information and surgical technique information for each of multiple distinct surgical cases. Using the training set, a learning algorithm trains a model to identify comparable surgical cases and/or to recommend particular techniques likely to maximize positive outcomes under the circumstances. Typically, the model is configured to receive as input surgical case patient information (e.g., mass details, BMI, age, gender, etc.).

In some implementations of the process 300, the surgical planning system may learn based on surgical results. This learning may be used to identify patterns of factors that may provide a particular search results. To elaborate, a surgical planning system may learn via, e.g., machine learning or artificial intelligence, to identify factors that should be considered by the surgeon in developing a surgical plan. Factors include characteristics of the patient or his or her condition. Characteristics of the patient could include, for example, gender, age, comorbidity status, BMI, prior surgical history. Characteristics of his or her condition could include size of tumor, location of tumor, proximity of tumor to adjacent structures such as blood vessels or urinary collecting system, and the like. Based on current medical understanding, the learning system can identify many factors that the surgeons will want to consider in formulating a surgical plan. But for some surgeries or patient conditions there may be factors or correlations that the medical field has not yet understood to be important in deciding on a surgical approach. For example, there may be a correlation between BMI and location of tumor that should be heavily weighted in deciding between a robotic procedure and an open procedure.

In another example, the learned model recommends a specific surgical technique that optimizes an outcome, which may be defined in terms of success of surgical objectives, lack of complications, blood loss, etc. Some examples of key objectives for some surgery types include the following: negative margins (all tumor resection cases), negative margins combined with a maximum patient length of stay, negative margins combined with normal erectile function (prostatectomy), and long-term renal function. As these key objectives are optimized across a comparison of other surgical techniques, a specific surgical technique is recommended by the learned model.

An appropriate training algorithm may be used to train a machine learning model using training information such as prior surgical case information and the associated results. The training algorithm may be used to recognize patterns in the data points between the independent variables (input such as a patient's basic information, preoperative parameters, and/or aspects of a surgical technique employed on the patient) and the dependent variables (output such as postoperative information or other information about the result of the surgery) so as to accurately predict information (new output) when presented with a new patient information (new input). The training algorithm may be based on any one of several machine learning algorithms. Machine learning algorithms can be divided into three broad categories: supervised learning, unsupervised learning, and reinforcement learning.

Supervised learning is useful where an output (such as postoperative information or other information about the result of the surgery) is available for a training dataset (e.g., a plurality of surgical cases). In supervised learning, the output is sometimes referred to as labels. Examples of machine learning algorithms that are supervised include but are not limited to linear regression, logistic regression, decision trees, support vector machine (SVM), naive Bayes, k-nearest neighbors, and neural networks (multilayer perceptron). Semi-supervised learning is a type of supervised learning having a small amount of labeled data and a large amount of unlabeled data for a certain dataset. Unsupervised learning is useful where implicit relationships in a given unlabeled dataset (output values are not pre-assigned) have not been discovered. An example of a machine learning algorithm that is unsupervised includes k-means. Reinforcement learning falls between supervised and unsupervised learning, where some feedback is available for each predictive step or action but there is no precise label. Rather than being presented with correct input/output pairs as in supervised learning, a given input is mapped to a reward function that an agent is trying to maximize. An example of a machine learning algorithm that is reinforcement-based includes a Markov Decision Process. Other types of learning that may fall into the one or more of the categories described above include, for example, deep learning and artificial neural networks (e.g., convolutional neural networks).

Various training tools or frameworks may exist to train the machine learning model. Examples of proprietary training tools include but are not limited to Amazon Machine Learning, Microsoft Azure Machine Learning Studio, DistBelief, Microsoft Cognitive Toolkit. Examples of open source training tools include but are not limited to Apache Singa, Caffe, H2O, PyTorch, MLPACK, Google TensorFlow, Torch, and Accord.Net.

Training a machine learning model may identify previously unrecognized parameters or groups of parameters that are important to surgical outcomes such as operative time, blood loss, ischemia time, intraoperative complications, mortality, and/or other parameters identified herein as postoperative information or patient follow-up information. And a trained machine learning model may be used to recommend a desired surgical plan. The trained machine learning model may take a patient's basic information and/or preoperative parameters as inputs and provide a recommended surgical plan as an output.

The trained machine learning model may take one of several forms. In some implementations, the trained machine learning model is a classification and regression tree or a random forest tree. In some implementations, the trained machine learning model is an artificial neural network, such as a convolutional neural network. In some implementations, the trained machine learning model is a linear classifier, such as a linear regression, logistic regression, or support vector machine.

### VII. Examples

The following hypothetical example is provided to illustrate certain embodiments. A surgery is being planned for patient John Doe. Mr. Doe is 62 years old and has been diagnosed with a tumor in his left kidney. The tumor measures 4 centimeters in diameter, is located entirely within the kidney (100% endophytic) in the lower pole, and has an anterior orientation. The complexity of the case, as measured by the nephrometry score, is 7a.

In formulating the surgical plan, the surgeon will make a number of decisions to arrive at a planned surgical technique. Examples include: (i) surgery type: should this be done as a partial nephrectomy or a radical nephrectomy; (ii) surgical procedure: should this be done as an open, laparoscopic, or robotic-assisted laparoscopic procedure; and (iii) surgical approach: if surgery type is determined to be a partial nephrectomy, should the kidney be approached from the front (transperitoneal approach) or the rear (retroperitoneal approach).

The surgeon utilizes the surgical planning system in order to obtain information regarding comparable cases and, potentially, recommendations regarding a proposed surgical technique. The method in which this information is generated and delivered to the surgeon may occur as described in the following embodiments.

In some embodiments, the surgeon runs a manual query using the system to obtain a list of comparable cases (including associated case profiles). This may involve the surgeon initiating a query using parameters defined by the system administrators or coded in the surgical planning system. For example, the surgical planning system may set the parameters to be gender, age, size of tumor, location of tumor, orientation of tumor, and nephrometry score. The surgeon inputs each of these parameters for Mr. Doe's case, and the system generates a list of cases that match all of the parameters, either exactly or within some pre-defined range (e.g., the comparable cases involve patients of age 62-65). As an example, the surgical planning system returns ten comparable cases, some presenting radical laparoscopic nephrectomy surgeries and their results, others presenting robotic-assisted laparoscopic partial nephrectomy surgeries and their results, and still others presenting open partial nephrectomy surgeries and their results.

In certain embodiments, the surgeon need not manually run a search query in order to generate a list of comparable cases. Rather, the system automatically generates such list by applying algorithms developed by the system administrators (and executed using a surgical planning system or related tool), utilizing distance functions, to provide information regarding Mr. Doe that is known preoperatively (such as his age, gender and the characteristics of his tumor) and comparing this information to similar information for all other cases available for searching in the system. In some cases, the available pre-surgical information relating to Mr. Doe includes images and/or three-dimensional models derived from such images.

In such embodiments, the system may, in addition to generating a list of comparable cases, provide aggregated analytics regarding such cases. For example, for cases that are comparable to Mr. Doe's, information regarding the most common surgical technique; or, which surgical technique resulted in the lowest percentage of complications, or the shortest patient hospital stay.

In certain embodiments, surgical planning systems employ a model or other feature derived or tuned using machine learning. The machine learning may improve the relevance of the resulting information, and the system may also generate recommendations regarding a surgical technique. Such recommendations could take many forms, including text, diagrams and/or scores.

As an example of how machine learning can improve searching for comparable cases, an initial distance function may not have considered the patient's body mass index as an important factor in deciding surgical approach. Over time, the system—via machine learning—detects that the complication rates are abnormally high when the patients are obese and the surgeons performed a robotic-assisted procedure, using a retroperitoneal approach. A learning approach may modify the surgical planning system to include BMI as a factor in determining comparable cases and/or in scoring proposed surgical plans. In some cases, to improve the search for comparable cases, the system weight towards BMI more heavily (compared to an initial search or distance function) when generating the scores associated with a retroperitoneal approach and transperitoneal approach for, e.g., Mr. Doe.

### VIII. Other Embodiments

The various processes, algorithms, software, etc. disclosed herein may be expressed (or represented) as data and/or instructions embodied in various computer-readable media, in terms of their behavioral, logical, and/or other characteristics. Computer-readable media in which such formatted data and/or instructions may be embodied include, but are not limited to, non-volatile storage media in various forms (e.g., optical, magnetic or semiconductor storage media). The computer-readable media corresponding to such systems are also intended to fall within the scope of the present disclosure.

Transfers of such data and/or instructions include, but are not limited to, transfers (uploads, downloads, e-mail, etc.) over the Internet and/or other computer networks via one or more data transfer protocols (e.g., HTTP, FTP, SMTP, etc.). When received within a computer system via one or more computer-readable media, such data and/or instruction-based expressions of the described surgical planning systems and related systems may be processed by a processing entity (e.g., one or more processors) within the computer system in conjunction with execution of one or more other computer programs.

Reference herein to “one embodiment” or “an embodiment” means that a particular feature, structure, or characteristic described in connection with the embodiment can be included in one some or all of the embodiments of the present disclosure. The usages or appearances of the phrase “in one embodiment” or “in another embodiment” in the specification are not referring to the same embodiment, nor are separate or alternative embodiments necessarily mutually exclusive of one or more other embodiments. The same applies to the term “implementation.” The present disclosure is neither limited to any single aspect nor embodiment thereof, nor to any combinations and/or permutations of such aspects and/or embodiments. Moreover, each of the aspects of the present disclosure, and/or embodiments thereof, may be employed alone or in combination with one or more of the other aspects of the present disclosure and/or embodiments thereof. For the sake of brevity, certain permutations and combinations are not discussed and/or illustrated separately herein.

Further, an embodiment or implementation described herein as exemplary is not to be construed as preferred or advantageous, for example, over other embodiments or implementations; rather, it is intended to convey or indicate that the embodiment or the embodiments are example embodiment(s).

In the claims, the term “determine” and other forms thereof (i.e., determining, determined and the like or calculating, calculated and the like) means, among other things, calculate, assesses, determine and/or estimate and other forms thereof.

In addition, the terms “first,” “second,” and the like, herein do not denote any order, quantity, or importance, but rather are used to distinguish one element from another. Moreover, the terms “a” and “an” herein do not denote a limitation of quantity, but rather denote the presence of at least one of the referenced item. Further, the terms “data” and “metadata” may mean, among other things information, whether in analog or a digital form (which may be a single bit (or the like) or multiple bits (or the like)).

As used in the claims, the terms “comprises,” “comprising,” “includes,” “including,” “have,” and “having” or any other variation thereof, are intended to cover a non-exclusive inclusion, such that a process, method, article, or apparatus that comprises a list of elements does not include only those elements but may include other elements not expressly listed or inherent to such process, method, article, or apparatus.

The claim elements that do not recite “means” or “step” are not in “means plus function” or “step plus function” form. (See, 35 USC § 112(f)). Applicant's intend that only claim elements reciting “means” or “step” be interpreted under or in accordance with 35 U.S.C. § 112(f).

In the foregoing description, numerous specific details are set forth to provide a thorough understanding of the presented implementations. The disclosed implementations may be practiced without some or all of these specific details. In other instances, well-known process operations have not been described in detail to not unnecessarily obscure the disclosed implementations. While the disclosed implementations are described in conjunction with the specific implementations, it will be understood that it is not intended to limit the disclosed implementations.

Although the foregoing embodiments have been described in some detail for purposes of clarity of understanding, it will be apparent that certain changes and modifications may be practiced within the scope of the appended claims. It should be noted that there are many alternative ways of implementing the processes, systems, and apparatus of the present embodiments. Accordingly, the present embodiments are to be considered as illustrative and not restrictive, and the embodiments are not to be limited to the details given herein.

What is claimed is:

1. A surgical planning system for identifying surgical cases comparable to a surgery to be performed on a patient, the surgical planning system comprising a database system configurable to:
receive, from a computing device, (i) patient information about the patient and/or (ii) an image showing a region of the patient where the surgery is to be performed;
access a plurality of database records each including a case profile associated with a surgical case; and
identify one or more comparable cases, from among the case profiles, wherein the one or more comparable cases identify one or more surgical techniques and resulting outcomes found in case profiles having similar characteristics with the patient information about the patient and the image showing the region of the patient where the surgery is to be performed.

2. The system of claim 1, wherein the database system is further configurable to:
cause the computing device to present, via a user interface, analytics of the one or more comparable cases based at least in part on the one or more surgical techniques and the resulting outcomes associated with the one or more comparable cases.

3. The system of claim 1, wherein the database system is further configurable to:
cause the computing device to present, via a user interface, a recommendation of a surgical technique based at least in part on an analysis of the one or more surgical techniques identified in the one or more comparable cases and the resulting outcomes associated with the one or more comparable cases.

4. The system of claim 3, wherein the database system is further configurable to:
receive, from a user input, an indication of a selection of a recommended surgical technique for the surgery to be performed on the patient; and
train a machine learning algorithm of the surgical planning system for presenting a recommended surgical technique.

5. The system of claim 1, wherein the database system is further configurable to:
receive, from a user input, an indication of a selection of a comparable case to the surgery to be performed on the patient; and
train a machine learning algorithm of the surgical planning system for identifying comparable cases.

6. The system of claim 1, wherein the database system configurable to identify one or more comparable cases is configurable to:
determine, using a distance function, that the one or more comparable cases have case profiles that are similar to the patient information about the patient and the image showing the region of the patient where the surgery is to be performed.

7. The system of claim 1, wherein the surgical techniques each include a description of at least a surgery type, surgical procedure, surgical approach, and secondary surgical decisions in performing a surgical operation.

8. The system of claim 1, wherein the image showing the region where surgery is to be performed includes a medical image of a computed tomography (CT) scan, a magnetic imaging resonance (MRI) scan, an X-ray image, a positron emission tomography (PET) scan, an ultrasound image, and/or a three-dimensional reconstruction of the foregoing.

9. The system of claim 1, wherein the patient information about the patient includes patient age, gender, weight, height, race, body mass index (BMI), comorbidity status, prior surgical history, or combinations thereof.

10. The system of claim 9, wherein the patient information further includes tumor size, tumor location, tumor orientation, tumor proximity to organs and/or tissue, tumor growth pattern, or combinations thereof.

11. The system of claim 1, wherein the case profile includes for each database record postoperative information and patient follow-up information, wherein the postoperative information and patient follow-up information include: operative time, blood loss, ischemia time, complications, readmissions, length-of-stay in hospital after surgery, positive margins, postoperative erectile function, postoperative continence, renal volume, renal function, or combinations thereof.

12. A method for identifying surgical cases comparable to a surgery to be performed on a patient, the method comprising:
receiving, from a computing device, (i) patient information about the patient and/or (ii) an image showing a region of the patient where the surgery is to be performed;
accessing a plurality of database records each including a case profile associated with a surgical case; and
identifying one or more comparable cases, from among the case profiles, wherein the one or more comparable cases identify one or more surgical techniques and resulting outcomes found in case profiles having similar characteristics with the patient information about the patient and the image showing the region of the patient where the surgery is to be performed.

13. The method of claim 12, further comprising:
causing the computing device to present, via a user interface, analytics of the one or more comparable cases based at least in part on the one or more surgical techniques and the resulting outcomes associated with the one or more comparable cases.

14. The method of claim 12, further comprising:
causing the computing device to present, via a user interface, a recommendation of a surgical technique based at least in part on an analysis of the one or more surgical techniques identified in the one or more comparable cases and the resulting outcomes associated with the one or more comparable cases.

15. The method of claim 12, further comprising:
receiving, from a user input, an indication of a selection of a comparable case or a recommended surgical technique to the surgery to be performed on the patient; and
training a machine learning algorithm of the surgical planning system.

16. The method of claim 12, wherein identifying the one or more comparable cases includes:
determining, using a distance function, that the one or more comparable cases have case profiles that are similar to the patient information about the patient and the image showing the region of the patient where the surgery is to be performed.

17. The method of claim 12, wherein the surgical techniques each include a description of at least a surgery type, surgical procedure, surgical approach, and secondary surgical decisions in performing a surgical operation.

18. The method of claim 12, wherein the image showing the region where surgery is to be performed includes a medical image of a computed tomography (CT) scan, a magnetic imaging resonance (MRI) scan, an X-ray image, a positron emission tomography (PET) scan, an ultrasound image, and/or a three-dimensional reconstruction of the foregoing.

19. The method of claim 12, wherein the patient information about the patient includes patient age, gender, weight, height, race, body mass index, comorbidity status, prior surgical history, tumor size, tumor location, tumor orientation, tumor proximity to organs and/or tissue, tumor growth pattern, or combinations thereof.

20. The method of claim 12, wherein the case profile includes for each database record includes postoperative information and patient follow-up information, wherein the postoperative information and patient follow-up information includes: operative time, blood loss, ischemia time, complications, readmissions, length-of-stay in hospital after surgery, positive margins, postoperative erectile function, postoperative continence, renal volume, renal function, or combinations thereof.
